(19) United States
(12) Patent Application Publication
(10) Pub. No.: US 2025/0313671 A1
(43) Pub. Date: Oct. 9, 2025

(54) A HARDWOOD-DERIVED CARBOHYDRATE COMPOSITION

(71) Applicant: UPM-KYMMENE CORPORATION, Helsinki (FI)

(72) Inventor: Juha TAMPER, Helsinki (FI)

(21) Appl. No.: 18/869,818

(22) PCT Filed: Jun. 12, 2023
(86) PCT No.: PCT/FI2023/050345

§ 371 Date: Nov. 26, 2024

(30) Foreign Application Priority Data
Jun. 13, 2022 (FI) 20225519

(51) Int. Cl. C08H 7/00; C07C 29/00; C07H 1/08; C07H 3/02; C08K 5/09; C08K 5/1535; C08K 5/1545; C12P 7/06

(52) U.S. Cl. CPC C08H 6/00; C07C 29/00; C07H 1/08; C07H 3/02; C08K 5/09; C08K 5/1535; C08K 5/1545; C12P 7/06

(57) ABSTRACT

A hardwood-derived carbohydrate composition comprising 88-99.75 weight-% monomeric sugars is disclosed. The monomeric sugars include 55-85 weight-% of monomeric xylose. The carbonyl content of the carbohydrate composition is 10-1500 μg/g based on the total dry matter content of the carbohydrate composition. Further is disclosed a method for producing the hardwood-derived carbohydrate composition and the use of the same.

### FIELD OF THE INVENTION

The present disclosure relates to a hardwood-derived carbohydrate composition comprising monomeric sugars. Further, the present disclosure relates to a method for producing the hardwood-derived carbohydrate composition. Further, the present disclosure relates to the use of the hardwood-derived carbohydrate composition.

### BACKGROUND OF THE INVENTION

Different methods are known for converting bio-based raw material, such as lignocellulosic biomass, into a liquid stream of various sugars. Being able to provide a sufficiently pure carbohydrate composition with properties suitable for further applications, such a production of glycols, ethanol, or xylitol, has still remained as a task for researchers.

### SUMMARY

A hardwood-derived carbohydrate composition is disclosed. The hardwood-derived carbohydrate composition comprises monomeric sugars in an amount of 88-99.75 weight-% based on the total dry matter content of the carbohydrate composition, wherein the monomeric sugars include monomeric xylose. The amount of monomeric xylose in the carbohydrate composition is 55-85 weight-% based on the total dry matter content of the carbohydrate composition. The carbonyl content of the carbohydrate composition is 10-1500 μg/g based on the total dry matter content of the carbohydrate composition.

Further is disclosed a method for producing the hardwood-derived carbohydrate composition as defined in the current application, wherein the method comprises:
i) providing a feedstock of hardwood-derived carbohydrates in the form of a liquid fraction having a total dry matter content of 7-13 weight-%;
ii) adjusting the pH of the feedstock of hardwood-derived carbohydrates to a pH-value of 2.2-3.0;
iii) subjecting the feedstock having a pH-value of 2.2-3.0 to evaporation until the total dry matter content of the feedstock is 45-55 weight-%;
iv) adjusting the pH of the evaporated feedstock to a pH-value of 5.5-7.5;
v) subjecting the feedstock having a pH-value of 5.5-7.5 to chromatographic treatment by using a strong acid cation-exchange resin;
vi) subjecting the chromatography treated feedstock to decolorization treatment;
vii) subjecting feedstock of hardwood-derived carbohydrates, subjected to decolorization treatment, to ion exchange treatment; and
viii) subjecting the ion exchange treated feedstock to evaporation until the total dry matter content of the feedstock is 30-80 weight-%;
to produce the hardwood-derived carbohydrate composition.

Further is disclosed the use of the hardwood-derived carbohydrate composition as disclosed in the current specification in a fermentation process, in a catalytic hydrogenation process to produce sugar alcohols and/or glycols, for the recovery of rare sugars, or for the production of a sweetener. Further is disclosed the use of the method.

### DETAILED DESCRIPTION

A hardwood-derived carbohydrate composition is disclosed. The hardwood-derived carbohydrate composition comprises monomeric sugars in an amount of 88-99.75 weight-% based on the total dry matter content of the carbohydrate composition, wherein the monomeric sugars include monomeric xylose. The amount of monomeric xylose in the carbohydrate composition is 55-85 weight-% based on the total dry matter content of the carbohydrate composition. The carbonyl content of the carbohydrate composition is 10-1500 μg/g based on the total dry matter content of the carbohydrate composition.

The hardwood-derived carbohydrate composition may be a liquid or in liquid form. The method as disclosed in the current specification may produce the hardwood-derived carbohydrate composition in liquid form.

Further is disclosed a method for producing the hardwood-derived carbohydrate composition as defined in the current application, wherein the method comprises:
i) providing a feedstock of hardwood-derived carbohydrates in the form of a liquid fraction having a total dry matter content of 7-13 weight-%;
ii) adjusting the pH of the feedstock of hardwood-derived carbohydrates to a pH-value of 2.2-3.0;
iii) subjecting the feedstock having a pH-value of 2.2-3.0 to evaporation until the total dry matter content of the feedstock is 45-55 weight-%;
iv) adjusting the pH of the evaporated feedstock to a pH-value of 5.5-7.5;
v) subjecting the feedstock having a pH-value of 5.5-7.5 to chromatographic treatment by using a strong acid cation-exchange resin;
vi) subjecting the chromatography treated feedstock to decolorization treatment;
vii) subjecting feedstock of hardwood-derived carbohydrates, subjected to decolorization treatment, to ion exchange treatment; and
viii) subjecting the chromatography treated feedstock to evaporation until the total dry matter content of the feedstock is 30-80 weight-%;
to produce the hardwood-derived carbohydrate composition.

In one embodiment, steps i), ii), iii), iv), v), vi), vii), and viii), are carried out one after the other in this order. In one embodiment, steps i), ii), iii), iv), v), vi), vii), and viii), are carried out one after the other in this order without additional step(s) taking place in between.

Further is disclosed the use of the hardwood-derived carbohydrate composition as disclosed in the current specification in a fermentation process, in a catalytic hydrogenation process to produce sugar alcohols and/or glycols, for the recovery of rare sugars, or for the production of a sweetener. The fermentation process may be e.g., ethanol fermentation or glycol fermentation. The catalytic process may comprise catalytic conversion for the production of e.g. glycols. Mannose, rhamnose, galactose, and arabinose may be mentioned as examples of rare sugars. The sweetener may be e.g., xylitol or xylose. In one embodiment, the production of a sweetener comprises crystallization of xylose from the hardwood-derived-carbohydrate composition. In one embodiment, the sweetener is xylose in crystalline or syrup form.

In one embodiment, the hardwood-derived carbohydrate composition is a sweetener composition. In one embodiment, the method for producing the hardwood-derived carbohydrate composition is a method for producing a sweetener composition.

Further, is disclosed the use of the method as disclosed in the current specification for reducing the amount of soluble lignin in the hardwood-derived carbohydrate composition in order to reduce precipitation of lignin during the storage and/or transportation of the hardwood-derived carbohydrate composition.

Further is disclosed a hardwood-derived carbohydrate composition obtainable by the method as disclosed in the current specification. In one embodiment, the hardwood-derived carbohydrate composition obtainable by the method as disclosed in the current specification is the hardwood-derived carbohydrate composition as disclosed in the current specification. I.e., the hardwood-derived carbohydrate composition disclosed in the current specification may be produced by the method as disclosed in the current specification.

The hardwood-derived carbohydrate composition may be a beechwood-derived carbohydrate composition, a birchwood-derived carbohydrate composition, an eucalyptus wood-derived carbohydrate composition, an aspen wood-derived carbohydrate composition, or the hardwood-derived carbohydrate composition may be a combination of these, or a combination of these together with other hardwood species. In one embodiment, the hardwood-derived carbohydrate composition is a beechwood-derived carbohydrate composition, a birchwood-derived carbohydrate composition, an eucalyptus wood-derived carbohydrate composition, or an aspen wood-derived carbohydrate composition. In one embodiment, the hardwood-derived carbohydrate composition is a beechwood-derived carbohydrate composition.

The hardwood-derived carbohydrate composition as disclosed in the current specification relates to a composition that comprises carbohydrates but may also in addition comprise additional components and/or elements e. g., as disclosed in the current specification. Thus, the “hardwood-derived carbohydrate composition” may be considered as a “hardwood-derived carbohydrate-containing composition” or a “hardwood-derived composition comprising carbohydrates”.

The amount of monomeric sugars, i.e. monomeric C5 sugars and monomeric C6 sugars as well as the amount of oligomeric sugars, i.e. oligomeric C5 sugars and oligomeric C6 sugars, may be determined both qualitatively and quantitatively by high-performance liquid chromatography (HPLC) by comparing to standard samples. Examples of analysis methods can be found in e.g. Sluiter, A., et al., “Determination of sugars, byproducts, and degradation products in liquid fraction process samples”, Technical Report, National Renewable Energy Laboratory, 2008, and Sluiter, A., et al., “Determination of Structural Carbohydrates and Lignin in Biomass”, Technical Report, National Renewable Energy Laboratory, revised 2012.

As used herein, any weight-percentages are given as percent of the total dry matter content of the carbohydrate composition unless specified otherwise. Similarly, other fractions of weight (ppm etc.) may also denote a fraction of the total dry matter content of the carbohydrate composition unless specified otherwise.

By the expression “C5 sugars” should be understood in this specification, unless otherwise stated, as referring to xylose, arabinose, or any mixture or combination thereof. By the expression “C6 sugars” should be understood in this specification, unless otherwise stated, as referring to glucose, galactose, mannose, or fructose, any mixture or combination thereof. By the expression that the sugar is “monomeric” should be understood in this specification, unless otherwise stated, as referring to a sugar molecule present as a monomer, i.e. not coupled or connected to any other sugar molecule(s).

In the current specification the amounts of different components/elements in the hardwood-derived carbohydrate composition are presented in weight-% based on the total dry matter content of the carbohydrate composition.

The expression “total dry matter content” may refer to the total amount of solids including soluble or dissolved solids. The hardwood-derived carbohydrate composition may be free of suspended solids, and contains only soluble solids.

In this specification the term “total dry matter content of the carbohydrate composition” may refer to the weight of the carbohydrate composition as determined after removing any solid particles or material from the carbohydrate composition, e.g. by filtering, and subjecting the filtrate to drying at a temperature of 45° C. for 24 hours. The effectiveness of the drying may be assured by weighing the sample, drying for a further two hours at the specified temperature, and reweighing the sample. If the measured weights are the same, the drying has been complete, and the total weight may be recorded.

As is clear to the skilled person, the total amount of the different components/elements in the hardwood-derived carbohydrate composition may not exceed 100 weight-%. The amount in weight-% of the different components/elements in the hardwood-derived carbohydrate composition may vary within the given ranges.

In one embodiment, the amount of monomeric xylose in the carbohydrate composition is 60-80 weight-%, or 62.5-75 weight-%, based on the total dry matter content of the carbohydrate composition.

In one embodiment, the carbonyl content of the carbohydrate composition is 15-1000 μg/g, or 20-750 μg/g, or 25-500 μg/g, or 30-300 μg/g, based on the total dry matter content of the carbohydrate composition. The expression “carbonyl content” may be taken as the content of carbonyl compounds comprising a functional group composed of a carbon atom double-bonded to an oxygen atom, i.e. C═O. A carbonyl group is common to several classes of organic compounds, as part of many larger functional groups. A compound containing a carbonyl group is often referred to as a carbonyl compound. Aldehydes, ketones, and carboxylic acids may be mentioned as examples containing a carbonyl group in their structure. The carbonyl in content the carbohydrate composition may be determined according to standard ASTM E411-05 (2009).

The hardwood-derived carbohydrate composition has the added utility of containing only a minor amount of carbonyl groups. As a result of this, e.g. fermentation of the carbohydrate composition proceeds more smoothly when there are less carbonyl groups present causing harmful side reactions.

In one embodiment, the carbohydrate composition exhibits an ICUMSA color value of 10-2500 IU, or 20-2000 IU, or 30-1500 IU, or 40-1000 IU, or 50-500 IU. The ICUMSA color value may be measured using a modified ICUMSA GS1 method without adjusting the pH of the sample to be analyzed and filtering the sample through a 0.45 μm filter before analysis. The measurement is conducted in room temperature and with the pH of the carbohydrate composition being 2.2-3.

In one embodiment, the carbohydrate composition comprises soluble lignin in an amount of 0.05-2.0 weight-%, or 0.1-1.5 weight-%, or 0.15-1.0 weight-%, or 0.20-0.5 weight-%, based on the total dry matter content of the carbohydrate composition. The presence of soluble lignin in the carbohydrate composition may evidence that the carbohydrate composition is derived from wood.

The amount of soluble lignin may be determined by UV-VIS absorption spectroscopy in the following manner: The amount of soluble lignin present in the carbohydrate composition is determined by diluting a sample of carbohydrate composition so that its absorbance at 205 nm is 0.2-0.7 AU when compared to a reference sample of pure water and using a cuvette with a path length of 1 cm. The soluble lignin content of the sample in mg/l may then be calculated using the following equation

$$x = ( \frac{A}{a} ) \times D$$

where A is absorbance of the sample, a is the absorptivity coefficient 0.110 l/mgcm, and D is a dilution factor.

The total dry matter content of the hardwood-derived carbohydrate composition may be 8-80 weight-%, or 15-75 weight-%, or 20-70 weight-% when determined after drying at a temperature of 45° C. for 24 hours.

In one embodiment, the conductivity of a 65% aqueous solution of the carbohydrate composition is 0.1-30 μS/cm, or 0.2-20 μS/cm, or 0.3-10 μS/cm, or 0.4-5 μS/cm, or 0.5-2.5 μS/cm, when determined according to SFS-EN 27888 (1994).

In one embodiment, the carbohydrate composition comprises rhamnose in an amount of 0.2-7 weight-%, or 0.4-5 weight-%, or 0.6-3.0 weight-%, based on the total dry matter content O the carbohydrate composition. The amount of rhamnose may be determined by high-performance anion-exchange chromatography with pulsed amperometric detection (HPAE-PAD).

In one embodiment, the carbohydrate composition comprises carboxylic acids in a total amount of at most 1.5 weight-%, or at most 1 weight-%, or at most 0.5 weight-%, or at most 0.25 weight-%, or at most 0.1 weight-%, based on the total dry matter content of the carbohydrate composition.

In one embodiment, the carbohydrate composition comprises monomeric sugars in a total amount of 88-99.75 weight-%, or 90-99.5 weight-%, or 92-99.25 weight-%, or 94-99 weight-%, based on the total dry matter content of the carbohydrate composition.

In one embodiment, the carbohydrate composition comprises monomeric sugars and oligomeric sugars in a total amount of 95-99.9 weight-%, or 96-99.8 weight-%, or 97-99.7 weight-%, or 98-99.6 weight-%, based on the total dry matter content of the carbohydrate composition. In one embodiment, the carbohydrate composition comprises oligomeric sugars in an amount of 0.1-9 weight-%, or 0.2-7 weight-%, or 0.3-5 weight-% based on the total dry matter content of the carbohydrate composition. The chromatographic treatment has the added utility of decreasing the amount of oligomeric sugars in the hardwood-derived carbohydrate composition.

By the expression that the sugar is “oligomeric” should be understood in this specification, unless otherwise stated, as referring to a sugar molecule consisting of two or more monomers coupled or connected to each other.

The oligomeric C5 sugars may be xylose and/or arabinose. The oligomeric C6 sugars may be glucose, galactose, mannose, fructose, and/or rhamnose.

In one embodiment, the carbohydrate composition comprises monomeric C6 sugars in an amount of 15-30 weight-%, or 18-28 weight-%, based on the total dry matter content of the carbohydrate composition.

In one embodiment, the monomeric sugars include monomeric glucose and monomeric xylose, and the weight ratio of monomeric glucose to monomeric xylose is 0.067-0.2, or 0.08-0.17, or 0.1-0.14. The inventors surprisingly found out that by the method as disclosed in the current specification, one is able to produce a hardwood-derived carbohydrate composition comprising a high content of monomeric C5 sugars and especially a high ratio of monomeric xylose compared to monomeric glucose. By the method as disclosed in the current specification, the C5 sugars may be efficiently recovered as a hardwood-derived carbohydrate composition.

The carbohydrate composition may comprise organic impurities (including soluble lignin) in an amount of at most 2 weight-%, or at most 1 weight-%, or at most 0.5 weight-%, or at most 0.25 weight-%, based on the total dry matter content of the carbohydrate composition.

Organic acids can be mentioned as examples of organic impurities. Non-limiting examples of organic impurities are oxalic acid, citric acid, succinic acid, formic acid, acetic acid, levulinic acid, 2-furoic acid, 5-hydroxymethylfurfural (5-HMF), furfural, glycolaldehyde, glyceraldehyde, as well as various acetates, formiates, and other salts or esters. The quality and quantity of organic impurities in the carbohydrate composition may be determined using e.g. a HPLC coupled with e.g. a suitable detector, infrared (IR) spectroscopy, ultraviolet-visible (UV-VIS) spectroscopy, or nuclear magnetic resonance (NMR) spectrometry.

The carbohydrate composition may comprise inorganic impurities. The carbohydrate composition may comprise inorganic impurities in an amount of 0-0.1 weight-%, or 0-0.05 weight-%, or 0-0.02 weight-%, based on the total dry matter content of the carbohydrate composition. The inorganic impurities may be e.g. a soluble inorganic compound in the form of various salts. The inorganic impurities may be salts of the group of elements consisting of Al, As, B, Ca, Cd, Cl, Co, Cr, Cu, Fe, K, Mg, Mn, Mo, Na, Ni, P, Pb, S, Se, Si, and Zn. The amounts of inorganic impurities in the carbohydrate composition can be analyzed using inductively coupled plasma-optical emission spectroscopy (ICP-OES) according to standard SFS-EN ISO 11885:2009. Alternatively, ion chromatography (IC) may be used.

The method for producing the hard-wood derived carbohydrate composition comprises providing a feedstock of hardwood-derived carbohydrates in the form of a liquid fraction having a total dry matter content of 7-13 weight-%. Such a feedstock of hardwood-derived carbohydrates may be provided e.g. in the following manner:
Firstly, a wood-based feedstock originating from wood-based raw material and comprising hardwood chips may be provided. Then the wood-based feedstock may be subjected to a pretreatment to form a slurry, wherein the pretreatment comprises:
subjecting the wood-based feedstock to an impregnation treatment with an impregnation liquid comprising sulphuric acid, wherein the impregnation treatment is carried out at a temperature of 40-100° C. for 1-30 minutes;
subjecting the impregnated wood-based feedstock to steam explosion treatment to form steam-treated wood-based feedstock, wherein the amount of sulphuric acid in the steam explosion treatment is 0.10-0.75 weight-% based on the total dry matter content of the wood-based feedstock;
mixing the steam-treated wood-based feedstock with a liquid to form the slurry; and
separating the slurry into a liquid fraction and a fraction comprising solid cellulose particles by a solid-liquid separation process to recover the liquid fraction as the feedstock of hardwood-derived carbohydrates.

By the expression “pretreating” or “pretreatment” should be understood in this specification, unless otherwise stated, (a) process(es) conducted to convert wood-based feedstock to a slurry which may be separated into a liquid fraction and a fraction comprising solid cellulose particles. I.e. the liquid fraction may be separated from the fraction comprising solid cellulose particles. The fraction comprising solid cellulose particles may further include an amount of lignocellulose particles as well as lignin particles in free form. Lignocellulose comprises lignin chemically bonded to the cellulose particles.

The wood-based raw material may originate e.g. from beech, birch, eucalyptus, ash, oak, maple, chestnut, willow, aspen, or poplar. The wood-based raw material may also be any combination or mixture of these.

In general, wood and wood-based raw materials are essentially composed of cellulose, hemicellulose, lignin, and extractives. Cellulose is a polysaccharide consisting of a chain of glucose units. Hemicellulose comprises polysaccharides, such as xylan, mannan, and glucan.

Providing the wood-based feedstock may comprise subjecting wood-based raw material to a mechanical treatment selected from debarking, chipping, dividing, cutting, beating, grinding, crushing, splitting, screening, and/or washing the wood-based raw material to form the wood-based feedstock. During the mechanical treatment e.g. wood logs can be debarked and/or wood chips of the specified size and structure can be formed. The formed wood chips can also be washed, e.g. with water, in order to remove e.g. sand, grit, and stone material therefrom. Further, the structure of the wood chips may be loosened before the pretreatment step. The wood-based feedstock may contain a certain amount of bark from the wood logs.

Providing the wood-based feedstock may comprise purchasing the wood-based feedstock. The purchased wood-based feedstock may comprise purchased wood chips or sawdust that originate from wood-based raw material.

Pretreatment of the wood-based feedstock may comprise one or more different pretreatment steps. During the different pretreatment steps the wood-based feedstock as such changes. The aim of the pretreatment step(s) is to form a slurry for further processing.

The pretreatment may comprise subjecting the wood-based feedstock to pre-steaming. The pretreatment may comprise subjecting the wood-based feedstock received from the mechanical treatment to pre-steaming. Pretreatment may comprise, before subjecting to the impregnation treatment, subjecting the wood-based feedstock to pre-steaming to form pre-steamed wood-based feedstock. The pretreatment may comprise, an impregnation treatment and a steam explosion treatment and comprise, before subjecting the wood-based feedstock to impregnation treatment and thereafter to steam explosion treatment, subjecting the wood-based feedstock to pre-steaming.

The pre-steaming of the wood-based feedstock may be carried out with steam having a temperature of 100-130° C., at atmospheric pressure. During the pre-steaming the wood-based feedstock is treated with steam of low pressure. The pre-steaming may be also carried out with steam having a temperature of below 100° C., or below 98° C., or below 95° C. The pre-steaming has the added utility of reducing or removing air from inside of the wood-based feedstock. The pre-steaming may take place in at least one pre-steaming reactor.

The pretreatment may comprise subjecting the wood-based feedstock to an impregnation treatment with an impregnation liquid comprising sulphuric acid. The impregnation liquid may consist of sulphuric acid and water. The impregnation liquid may comprise sulphuric acid in an amount of at most 20 weight-% based on the total weight of the impregnation liquid. Subjecting the wood-based feedstock to the impregnation treatment may form an impregnated wood-based feedstock comprising sulphuric acid in an amount of at least 0.5 weight-% based on the total dry matter content of the wood-based feedstock.

The impregnation treatment may be carried out to the wood-based feedstock received from the mechanical treatment and/or from the pre-steaming. The wood-based feedstock may be transferred from the mechanical treatment and/or from the pre-steaming to the impregnation treatment with a feeder. The feeder may be a screw feeder, such as a plug screw feeder. The feeder may compress the wood-based feedstock during the transfer. When the wood-based feedstock is then entering the impregnation treatment, it may become expanded and absorbs the impregnation liquid.

The sulphuric acid may be dilute sulphuric acid. The total amount of acid added to the wood-based feedstock may be 0.3-5.0% w/w, 0.5-3.0% w/w, 0.6-2.5% w/w, 0.7-1.9% w/w, or 1.0-1.6% w/w based on the total dry matter content of the wood-based feed-stock. The impregnation liquid may act as a catalyst in affecting the hydrolysis of the hemicellulose in the wood-based feedstock. In one embodiment, the sulphuric acid catalyzes the hydrolysis of the hemicellulose in the wood-based feedstock to monomeric sugars.

The impregnation treatment may be conducted in at least one impregnation reactor or vessel. In one embodiment, two or more impregnation reactors are used. The transfer from one impregnation reactor to another impregnation reactor may be carried out with a screw feeder.

The impregnation treatment may be carried out by conveying the wood-based feedstock through at least one impregnation reactor that is at least partly filled with the impregnation liquid, i.e. the wood-based feed-stock may be transferred into the impregnation reactor, where it sinks into the impregnation liquid, and transferred out of the impregnation reactor such that the wood-based feedstock is homogenously impregnated with the impregnation liquid. As a result of the impregnation treatment, impregnated wood-based feedstock is formed. The impregnation treatment may be carried out as a batch process or in a continuous manner.

The residence time of the wood-based feedstock in an impregnation reactor, i.e. the time during which the wood-based feedstock is in contact with the impregnation liquid, may be 1-30 minutes. The temperature of the impregnation liquid may be e.g. 20-99° C., or 40-95° C., or 60-93° C. Keeping the temperature of the impregnation liquid below 100° C. has the added utility of hindering or reducing hemicellulose from dissolving. In one embodiment, the impregnation treatment is carried out at a temperature of 80-100° C., or 90-99° C., for 1-30 minutes.

After the impregnation treatment, the impregnated wood-based feedstock may be allowed to stay in e.g. a storage tank or a silo for a predetermined period of time to allow the impregnation liquid absorbed into the wood-based feedstock to stabilize. This predetermined period of time may be 15-60 minutes, or e.g. about 30 minutes.

In one embodiment, the wood-based feedstock is subjected to an impregnation treatment with dilute sulphuric acid having a concentration of 1.32% w/w and a temperature of 92° C.

Pretreatment may comprise subjecting the wood-based feedstock to steam explosion treatment. The wood-based feedstock from the impregnation treatment may be subjected to steam explosion treatment. I.e. pretreatment may comprise subjecting the impregnated wood-based feedstock to steam explosion treatment to form a steam-treated wood-based feedstock.

The pretreatment may thus comprise mechanical treatment of wood-based material to form a wood-based feedstock, the pre-steaming of the wood-based feedstock to form pre-steamed feedstock, impregnation treatment of the pre-steamed wood-based feedstock to form impregnated wood-based feedstock, and the steam explosion treatment of the impregnated wood-based feedstock. In one embodiment, the pretreatment in ii) comprises pre-steaming the wood-based feedstock, impregnation treatment of the pre-steamed wood-based feedstock, and steam explosion treatment of the impregnated wood-based feed-stock. In one embodiment, the pretreatment in ii) comprises impregnation treatment of the wood-based feed-stock, and steam explosion treatment of the impregnated wood-based feedstock. I.e. the wood-based feedstock having been subjected to the impregnation treatment may thereafter be subjected to the steam explosion treatment. Also, the wood-based feedstock having been subjected to pre-steaming, may then be subjected to the impregnation treatment and thereafter the impregnated wood-based feedstock having been subjected to the impregnation treatment may be subjected to steam explosion treatment.

The wood-based feedstock can be stored in e.g. chip bins or silos between the different treatments. Alternatively, the wood-based feedstock may be conveyed from one treatment to the other in a continuous manner.

The pretreatment may comprise subjecting the impregnated wood-based feedstock to steam explosion treatment to form steam-treated wood-based feedstock. The amount of sulphuric acid in the steam explosion treatment may be 0.10-0.75 weight-% based on the total dry matter content of the wood-based feedstock. The steam explosion treatment may be carried out by treating the impregnated wood-based feedstock with steam having a temperature of 130-240° C., or 180-200° C., or 185-195° C. under a pressure of 0.17-3.25 MPaG followed by a sudden, explosive decompression of the feedstock. The feedstock may be treated with the steam for 1-20 minutes, or 1-18 minutes, or 2-15 minutes, or 4-13 minutes, or 3-10 minutes, or 3-8 minutes, before the sudden, explosive decompression of the steam-treated wood-based feedstock.

In this specification, the term “steam explosion treatment” may refer to a process of hemihydrolysis in which the feedstock is treated in a reactor (steam explosion reactor) with steam having a temperature of 130-240° C., or 180-200° C., or 185-195° C. under a pressure of 0.17-3.25 MPaG followed by a sudden, explosive decompression of the feedstock that results in the rupture of the fiber structure of the feedstock.

In one embodiment, the amount of sulphuric acid in the steam explosion treatment may be 0.10-0.75 weight-% based on the total dry matter content of the wood-based feedstock. The amount of acid present in the steam explosion treatment may be determined by measuring the sulphur content of the liquid of the steam-treated wood-based feedstock or the liquid part of the steam-treated wood-based feedstock after steam explosion treatment. The amount of sulphuric acid in the steam explosion reactor may be determined by subtracting the amount of sulphur in the wood-based feedstock from the measured amount of total sulphur in the steam-treated wood-based feedstock.

The steam explosion treatment may be conducted in a pressurized reactor. The steam explosion treatment may be carried out in the pressurized reactor by treating the impregnated wood-based feedstock with steam having a temperature of 130-240° C., or 180-200° C., or 185-195° C. under a pressure of 0.17-3.25 MPaG followed by a sudden, explosive decompression of the—feedstock. The impregnated wood-based feedstock may be introduced into the pressurized reactor with a compressing conveyor, e.g. a screw feeder. During transportation with the screw feeder, if used, the acid in liquid form is removed, and a part of the impregnation liquid absorbed by the feedstock is removed as a pressate while most of it remains in the feedstock. The impregnated wood-based feedstock may be introduced into the pressurized reactor along with steam and/or gas. The pressure of the pressurized reactor can be controlled by the addition of steam. The pressurized reactor may operate in a continuous manner or as a batch process. The impregnated wood-based feedstock, e.g. the wood-based feedstock that has been subjected to an impregnation treatment, may be introduced into the pressurized reactor at a temperature of 25-140° C. The residence time of the feedstock in the pressurized reactor may be 0.5-120 minutes. The term “residence time” should in this specification, unless otherwise stated, be understood as the time between the feedstock being introduced into or entering e.g. the pressurized reactor and the feed-stock being exited or discharged from the same.

As a result of the hemihydrolysis of the wood-based feedstock affected by the steam explosion treatment in the reactor, the hemicellulose present in the wood-based feedstock may become hydrolyzed or degraded into e.g. xylose oligomers and/or monomers. The hemicellulose comprises polysaccharides such as xylan, mannan and glucan. Xylan is thus hydrolyzed into xylose that is a monosaccharide. In one embodiment, 87-95%, or 89-93%, or 90-92%, of xylan present the impregnated wood-based feedstock is converted into xylose.

Thus, steam explosion of the feedstock may result in the formation of an output stream. The output stream from the steam explosion may be subjected to steam separation. The output stream from the steam explosion may be mixed or combined with a liquid, e.g. water. The output stream of the steam explosion may be mixed with a liquid to form a slurry. The liquid may be pure water or water containing C5 sugars. The water containing C5 sugars may be recycled water from separation and/or washing the fraction comprising solid cellulose particles before enzymatic hydrolysis. The output stream may be mixed with the liquid and the resulting mass may be homogenized mechanically to break up agglomerates. Pretreatment may comprise mixing the steam- treated wood-based feedstock with a liquid to form the slurry.

Thus, as a result of the pretreatment a slurry may thus be formed. The slurry may comprise a liquid phase and a solid phase. The slurry may comprise solid cellulose particles. The slurry may be separated into a liquid fraction and a fraction comprising solid cellulose particles.

The method may comprise separating a liquid fraction and a fraction comprising solid cellulose particles by a solid-liquid separation process to recover the liquid fraction as feedstock of hardwood-derived carbohydrates. The solid-liquid separation process may comprise washing. The washing may be continued until the amount of soluble organic components in the fraction comprising solid cellulose particles is 0.5-5 weight-%, or 1-4 weight-%, or 1.5-3 weight-% based on the total dry matter content.

Separating the liquid fraction and the fraction comprising solid cellulose particles may be carried out by displacement washing or countercurrent washing. Thus, the solid-liquid separation process may be selected from displacement washing and countercurrent washing.

Displacement washing, or replacement washing as it may also be called, is a method for separating solids and liquid from each other by the use of a rather minor amount of washing liquid. Thus, displacement washing may be considered as an operation by which it is possible to wash solid particles with a minimum amount of washing liquid, such as water.

In countercurrent washing, the movement of the fraction comprising solid cellulose particles in generally in a forward direction, whereas the washing liquid, such as water, flows in the opposite direction. As for the displacement washing, also the countercurrent washing may reduce the consumption of washing liquid to a great extent.

The countercurrent washing may comprise at least two solid-liquid separation steps and one dilution in between the steps with washing solution. The washing solution may be clean water. The amount of water needed may vary depending on how many solid-liquid separation steps are performed in total, the total dry matter content in the feed of the solid-liquid separation step and the total dry matter content in the fraction comprising solid cellulose particles after each solid-liquid separation step.

The washing liquid may be fresh washing water or recycled washing water. The washing water may be fresh water, drinking water, or a sugar containing liquid with low sugar content. The conductivity of the washing liquid may be about 0.1 mS/cm.

The ratio of the used washing liquid to the solids may be 0.5:1-8:1 (w/w), or 0.5:1-5:1 (w/w), or 0.5:1-3:1 (w/w), or 0.5:1-2:1 (w/w) in the case of displacement washing. The ratio of the used washing liquid to the solids may be 0.5:1-8:1 (w/w), or 0.5:1-5:1 (w/w) in the case of countercurrent washing.

The progression of the displacement washing as well as of the countercurrent washing may be monitored by measuring the conductivity of the liquid fraction recovered from this treatment. Once the conductivity of the liquid fraction is below or equal to a predetermined threshold value of 0.35 mS/cm, one may conclude that that the desired amount of the C5 sugars and other soluble impurities have been removed from the fraction comprising solid cellulose particles and the washing may be concluded. In one embodiment, the washing is continued until the conductivity of the liquid fraction is 0.1-1.0 mS/cm or 0.2-0.5 mS/cm.

Alternatively, the separation may be carried out by filtration, decanting, and/or by centrifugal treatment. The filtration may be vacuum filtration, filtration based on the use of reduced pressure, filtration based on the use of overpressure, or filter pressing. The decanting may be repeated in order to improve separation.

The above described separation and/or washing may include recirculation of the e.g. the washing liquid in order to concentrate the liquid fraction, i.e. the feedstock of hardwood-derived carbohydrates, if needed, to provide the feedstock of hardwood-derived carbohydrates in the form of a liquid fraction having a total dry matter content of 7-13 weight-%.

Thus the method for producing a hardwood-derived carbohydrate composition comprises the step of providing a feedstock of hardwood-derived carbohydrates in the form of a liquid fraction having a total dry matter content of 7-13 weight-%.

The feedstock of hardwood-derived carbohydrates may comprise monomeric sugars in an amount of 50-80 weight-% based on the total dry matter content of the feedstock. The amount of monomeric xylose in the feedstock may be 40-60 weight-%. In the feedstock of hardwood-derived carbohydrates the weight ratio of monomeric glucose to monomeric xylose may be 0.067-0.2. The feedstock of hardwood-derived carbohydrates may comprise soluble lignin in an amount of 5-15 weight-% based on the total dry matter content of the feedstock. The feedstock of hardwood-derived carbohydrates may comprise organic impurities in an amount of 6-30 weight-% based on the total dry matter content of the feedstock. The feedstock of hardwood-derived carbohydrates may comprise carboxylic acids in an amount of 5-20 weight-% based on the total dry matter content of the feedstock. The feedstock of hardwood-derived carbohydrates may comprise inorganic impurities in an amount of 0-6 weight-%, or 0.1-3 weight-%, or 0.2-2 weight-%, or 0.3-1 weight-%, based on the total dry matter content of the feedstock.

The pH of the feedstock of hardwood-derived carbohydrates may then be adjusted to a pH-value of 2.2-3.0. The pH-value may be adjusted by using e.g. sodium hydroxide, potassium hydroxide, or the like. Adjusting the pH-value before evaporation to a value of 2.2-3.0 has the added utility of reducing or preventing lignin possibly present in the feedstock to precipitate during the evaporation. Further, by adjusting the pH-value it may be ensured that the organic acids possibly present in the feedstock are removed with the condensate.

Then the feedstock having a pH-value of 2.2-3.0 may be subjected to evaporation. The evaporation may be carried out by using a steam having a temperature of 75-85° C., or 77-83° C., or at about 79° C., in vacuum. The temperature of the feedstock may be 65-70° C., or 67-69° C., during the evaporation. The evaporation may be continued until the total dry matter content of the evaporated feedstock is 45-55 weight.

After the evaporation the pH of the evaporated feedstock is adjusted to a pH-value of 5.5-7.5. This pH adjustment has the added utility of assisting to carry out the chromatographic treatment in an efficient manner.

Then the evaporated and pH adjusted feedstock may be subjected to chromatographic treatment by using a strong acid cation-exchange resin.

The strong acidic cation-exchange resin is a bead-like product which has a sulfonic acid group in the cross-linked styrene frame. The strong acid cation-exchange resin may be in Na⁺ form or in H⁺ form. In one embodiment, the strong acid cation-exchange resin is in the Na⁺ form. The strong acid cation-exchange resin in the Na⁺ form may have a polystyrene structure whereto divinyl benzene groups are crosslinked. The average particle size may be 350 μm. Sulfonic acid may function as the functional group.

In one embodiment, the chromatographic treatment is carried out with a simulated moving bed (SMB) chromatography, or with any variation of simulated moving bed chromatography. Intermitted simulated moving bed chromatography (ISMB) and smart simulated moving bed chromatography (SSMB) may be mentioned as examples of such variations.

In one embodiment, the chromatographic treatment is carried out as a one stage process. I.e. the feedstock of hardwood-derived carbohydrates is subjected to the chromatographic treatment one time before continuing to the following step. The one stage chromatographic treatment may be carried out by using one or several consecutive columns. The flow rate through the column or columns may be 1-3 bed volumes per hour. Water may be used as an elution solution.

The chromatographic treatment has the added utility of e.g. reducing the color value caused by the lignin present and decreasing the amount of organic salts, inorganic salts, and metals, in the hardwood-derived carbohydrate composition. The purpose of the chromatographic treatment is not to fractionate or to separate the sugars of the feedstock but to remove unwanted components.

After the chromatographic treatment, the chromatographically treated feedstock may be subjected to decolorization treatment. In one embodiment, the decolorization treatment is carried out by subjecting the feedstock to anion exchange treatment, to filtration with a membrane, or to granular activated carbon treatment, or to any combination thereof. Granular activated carbon, or granular active carbon as it may also be called, may be considered as activated carbon being retained on a 50-mesh sieve. The particle size of the granular activated carbon may be 0.2-2 mm, or 0.3-1.5 mm. These have the added utility of removing soluble lignin affecting the colour of the feedstock.

The decolorized feedstock of hardwood-derived carbohydrates may then be subjected to ion exchange treatment.

In one embodiment, the ion exchange treatment comprises treating the feedstock with:
viia) cation exchange resin;
viib) strong anion exchange resin; and
viic) weak anion exchange resin,
in the order of first viia), then viib), and then viic).

The different type of ion exchange resins may be packed in separate columns. The flow direction of the feedstock through the columns follows the above order, first there is a cation exchange resin, then a strong anion exchange resin, and the weak anion exchange resin is the last ion exchange resin.

A regeneration solution may be used for regenerating the decolorization and ion exchange resins. A regenerating solution may be run through the decolorization and ion exchange resins at predetermined intervals. E.g. a sulphuric acid solution or a hydrochloric acid solution may be used to regenerate the cationic exchange resins. E.g. a sodium hydroxide solution may be used to regenerate the anionic exchange resins.

After the ion exchange treatment, the ion exchanged feedstock may be subjected to evaporation. This evaporation may also be referred to as the final evaporation. The evaporation may be carried out by using a steam having a temperature of 75-85° C., or 77-83° C., or at about 79° C., in vacuum. The temperature of the feedstock may be 65-70° C., or 67-69° C., during the evaporation.

The evaporation may be continued until the total dry matter content of the feedstock is 30-80 weight-%. The evaporation has the added utility of affecting the amount of organic acids that may be removed from the feedstock. Further, evaporating the feedstock until the total dry matter content of the feedstock is e.g. 60-70 weight-% has the added utility for being beneficial for storing and transporting the hardwood-derived carbohydrate composition.

The method as disclosed in the current specification has the added utility of providing a hardwood-derived carbohydrate composition with a high content of monomeric sugars, and especially monomeric xylose.

The method as disclosed in the current specification has the added utility of providing a hardwood-derived carbohydrate composition having a reduced amount of soluble lignin whereby the risk of the lignin precipitating during the storage and transportation of the hardwood-derived carbohydrate composition.

The method as disclosed in the current specification has also the added utility of providing a hardwood-derived carbohydrate composition having a reduced amount of inorganic impurities, e.g. metals.

The hardwood-derived carbohydrate composition has properties making it useful for e.g. ethanol fermentation or glycol fermentation. The hardwood-derived carbohydrate composition has the added utility of fulfilling purity properties required for use in catalytic hydrogenation process for the production of e.g. glycols or sugar alcohols. Further, the hardwood-derived carbohydrate composition may be used for recovering rare sugars, e.g. rhamnose. The hardwood-derived carbohydrate composition has the added utility of fulfilling purity properties required for further use in e.g. a process for producing a sweetener such as xylitol.

### EXAMPLES

Reference will now be made in detail to the embodiments of the present disclosure.

The description below discloses some embodiments in such a detail that a person skilled in the art is able to utilize the method based on the disclosure. Not all steps of the embodiments are discussed in detail, as many of the steps will be obvious for the person skilled in the art based on this disclosure.

### Example 1-Producing Hardwood-Derived Carbohydrate Composition

In this example a hardwood-derived carbohydrate composition was prepared.

First a wood-based feedstock comprising chips of beech wood was provided. The wood-based feedstock was then subjected to pretreatment in the following manner:

The wood-based feedstock was subjected to pre-steaming. Pre-steaming of the wood-based feedstock was carried out at atmospheric pressure with steam having a temperature of 100° C. for 180 minutes. The pre-steamed feedstock was then subjected to an impregnation treatment with dilute sulphuric acid having a concentration of 1.32% w/w and a temperature of 92° C. The pre-steamed wood-based feedstock was allowed to be affected by the impregnation liquid for 30 minutes. The acid-impregnated wood-based feedstock was then subjected to explosion treatment. The steam explosion treatment was carried out by treating the impregnated wood-based feedstock with steam having a temperature of 191° C. followed by a sudden, explosive decompression of the wood-based feedstock to atmospheric pressure. The amount of sulphuric acid in steam explosion reactor was 0.33 weight-% based on the total dry matter content of the wood-based feedstock. In the determination of the amount of sulphuric acid the sulphur content of wood was 0,02 weight-% based on the total dry matter content of the wood used.

In the pretreatment, the conversion of xylan in the wood-based feedstock into xylose was 91% and the ratio of solubilized glucose to solubilized xylose was 0.14 as determined by HPLC-RI as detailed below. The steam-treated wood-based feedstock was then mixed with water in a mixing vessel.

As a result of the above pretreatment steps, a slurry was formed. The slurry comprised a liquid fraction and a fraction comprising solid cellulose particles. The slurry was then separated into a liquid fraction and a fraction comprising solid cellulose particles by a solid-liquid separation process, which in this example was countercurrent washing. The countercurrent washing was continued until the amount of soluble components in the fraction comprising solid cellulose particles was 2.0 weight-% based on the total dry matter content. The dry solids content of the fraction comprising solid cellulose particles was 32 weight-% after the washing. The total dry matter content of the liquid fraction was 9 weight-%.

The liquid fraction was recovered as the feedstock of hardwood-derived carbohydrates. Then the pH of the feedstock of hardwood-derived carbohydrates was adjusted to a pH-value of 2.9 by using sodium hydroxide (NaOH). The feedstock was then evaporated until the total dry matter content of the feedstock was about 50 weight-%. The temperature of the steam used for the evaporation was 79° C. in vacuum. The temperature of the feedstock was 68° C. during the evaporation.

Then the pH of the evaporated feedstock was adjusted to a pH-value of 6.0 by using sodium hydroxide (NaOH).

The pH-adjusted feedstock was then subjected to a chromatographic treatment with a simulated moving bed (SMB) chromatograph by using the strong acid cation exchange resin in the Na⁺ form (average particle size 350 μm). The chromatographic treatment was carried out at a flow rate through the column of two bed volumes per hour by using a one column system.

Then, the feedstock was subjected to decolorization treatment by an anion exchange resin.

After the decolorization treatment the feedstock was subjected to ion exchange treatment by using firstly a cation exchange resin, then a strong anion exchange resin, and finally a weak anion exchange resin.

A regeneration solution was run after every 30^{th} bed volume during the decolorization treatment by using 1% NaOH solution and 10% NaCl solution. The regeneration of the ion exchange resins used in the ion exchange treatment was also run after every 30th bed volumes. A 5% sulphuric acid solution was used to regenerate the cationic exchange resin and a 5% sodium hydroxide solution was used for the anionic exchange resin. After regenerating, the resins were flushed with water before the feedstock was fed to the treatments.

After the ion exchange treatment the feedstock was evaporated until the total dry matter content of the feedstock was 65 weight-%. The temperature of the steam used for the evaporation was 79° C. in vacuum. As a result a hardwood-derived carbohydrate composition was formed.

The hardwood-derived carbohydrate composition recovered was analyzed by HPLC-RI using a Waters e2695 Alliance Separation module, a Waters 2998 Photodiode Array, and a Waters 2414 Refractive Index detector. Separation was achieved with a Bio-Rad Aminex HPX-87 column with dimensions 300 mm×7.8 mm equipped with Micro-Guard Deashing and Carbo-P guard columns in series. Ultrapure water was used as eluent.

The amount of oligomeric sugars in the sample was determined by hydrolyzing the oligomeric sugars into monomeric sugars using acid hydrolysis, analyzing the acid hydrolyzed sample using HPLC-RI, and comparing the result to those for samples for which the hydrolysis was not performed. By subtracting the amount of monomeric sugars in the untreated sample, the amount of oligomeric sugars was calculated.

The results are presented in the below table:

| | Measured value | |
|---|---|---|
| | Feedstock of | Hardwood-derived |
| | hardwood-derived | carbohydrate |
| | carbohydrates | composition |
| Monomeric sugars total | 68.9 | 97.8 |
| amount, HPLC-RI (wt-%) | | |
| Monomeric xylose, HPLC-RI | 52.1 | 73.7 |
| (wt-%) | | |
| Monomeric glucose, HPLC-RI | 6.9 | 9.4 |
| (wt-%) | | |
| Soluble lignin, (wt-%), | 6.3 | 0.16 |
| UV 205 | | |
| Conductivity (mS/cm), 65% | 3.1 (9% | 0.003 (65% |
| solution | solution) | solution) |
| Organic impurities (wt-%)* | 21.4 | 0.3 |
| Total carboxylic acids (wt-%) | 10.7 | 0.1 |
| Inorganic impurities (wt-%)** | 0.2 | 0.02 |
| ICUMSA colour value (IU) | 49308 | 207 |
| Carbonyl content (μg/g), | 17215 | 121 |
| ASTM E411-05 (2009) | | |
| Rhamnose (wt-%) , HPAE-PAD | 1.9 | 2.0 |
| Total dry matter content | 9.0 | 65 |
| (wt-%), 45° C. 24 h | | |

wt-% = Weight-% based on the total dry matter content
*This is the combined amount of lignin (UV-205), the organic impurities (organic acids + furan + aldehydes)

measured with HPLC-PDA, as well as formiate, acetate and lactate as measured with IC.
* * Metal ions measured with ICP-OES, chlorides, sulphates, and phosphates measured with IC.

It is obvious to a person skilled in the art that with the advancement of technology, the basic idea may be implemented in various ways. The embodiments are thus not limited to the examples described above; instead they may vary within the scope of the claims.

The embodiments described hereinbefore may be used in any combination with each other. Several of the embodiments may be combined together to form a further embodiment. A hardwood-derived carbohydrate composition, a method, or a use as disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

1. A hardwood-derived carbohydrate composition comprising;
monomeric sugars in an amount of 88-99.75 weight-% based on the total dry matter content of the carbohydrate composition, wherein the monomeric sugars include monomeric xylose, the amount of monomeric xylose in the carbohydrate composition being 55-85 weight-% based on the total dry matter content of the carbohydrate composition, and wherein the carbonyl content of the carbohydrate composition is 10-1500 μg/g based on the total dry matter content of the carbohydrate composition.

2. The hardwood-derived carbohydrate composition of claim 1, wherein the amount of monomeric xylose in the carbohydrate composition is 60-80 weight-%, based on the total dry matter content of the carbohydrate composition.

3. The hardwood-derived carbohydrate composition of claim 1, wherein the carbonyl content of the carbohydrate composition is 15-1000 μg/g, based on the total dry matter content of the carbohydrate composition.

4. The hardwood-derived carbohydrate composition of claim 1, wherein the carbohydrate composition exhibits an ICUMSA color value of 10-2500 IU.

5. The hardwood-derived carbohydrate composition of claim 1, wherein the carbohydrate composition comprises soluble lignin in an amount of 0.05-2.0 weight-%, based on the total dry matter content of the carbohydrate composition.

6. The hardwood-derived carbohydrate composition of claim 1, wherein the conductivity of a 65% aqueous solution of the carbohydrate composition is 0.1-30 μS/cm, when determined according to SFS-EN 27888.

7. The hardwood-derived carbohydrate composition of claim 1, wherein the carbohydrate composition comprises rhamnose in an amount of 0.2-7 weight-%, based on the total dry matter content of the carbohydrate composition.

8. The hardwood-derived carbohydrate composition of claim 1, wherein the carbohydrate composition comprises carboxylic acids in an amount of at most 1.5 weight-%, based on the total dry matter content of the carbohydrate composition.

9. A method for producing a hardwood-derived carbohydrate composition of, wherein the method comprises:
i) providing a feedstock of hardwood-derived carbohydrates in the form of a liquid fraction having a total dry matter content of 7-13 weight-%;
ii) adjusting the pH of the feedstock of hardwood-derived carbohydrates to a pH-value of 2.2-3.0;
iii) subjecting the feedstock having a pH-value of 2.2-3.0 to evaporation until the total dry matter content of the feedstock is 45-55 weight-%;
iv) adjusting the pH of the evaporated feedstock to a pH-value of 5.5-7.5;
v) subjecting the feedstock having a pH-value of 5.5-7.5 to chromatographic treatment by using a strong acid cation-exchange resin;
vi) subjecting the chromatography treated feedstock to decolorization treatment;
vii) subjecting feedstock of hardwood-derived carbohydrates, subjected to decolorization treatment, to ion exchange treatment; and
viii) subjecting the ion exchange treated feedstock to evaporation until the total dry matter content of the feedstock is 30-80 weight-%;
to produce the hardwood-derived carbohydrate composition.

10. The method of claim 9, wherein the decolorization treatment is carried out by subjecting the feedstock to anion exchange treatment, to filtration with a membrane, to granular activated carbon treatment, or to any combination thereof.

11. The method of claim 9, wherein the ion exchange treatment comprises treating the feedstock with:
viia) cation exchange resin;
viib) strong anion exchange resin; and
viic) weak anion exchange resin,
in the order of first viia), then viib), and then viic).

12. Use of the hardwood-derived carbohydrate composition of claim 1 in a fermentation process, in a catalytic hydrogenation process to produce sugar alcohols and/or glycols, for the recovery of rare sugars, or for the production of a sweetener.

13. The use of claim 12, wherein the use is for a fermentation process, the fermentation process being ethanol fermentation or glycol fermentation.

14. The use of claim 12, wherein the use is for the production of a sweetener, the production of a sweetener comprising crystallization of xylose from the hardwood-derived carbohydrate composition.

15. Use of the method of claim 9 for reducing the amount of soluble lignin in the hardwood-derived carbohydrates composition in order to reduce precipitation of lignin to during the storage and/or transportation of the hardwood-derived carbohydrate composition.

16. The method of claim 9, wherein the hardwood-derived carbohydrate composition comprising monomeric sugars in an amount of 88-99.75 weight-% based on the total dry matter content of the carbohydrate composition, wherein the monomeric sugars include monomeric xylose, the amount of monomeric xylose in the carbohydrate composition being 55-85 weight-% based on the total dry matter content of the carbohydrate composition, and wherein the carbonyl content of the carbohydrate composition is 10-1500 μg/g based on the total dry matter content of the carbohydrate composition.
